(19) United States
(12) Patent Application Publication
(10) Pub. No.: US 2017/0049414 A1
(43) Pub. Date: Feb. 23, 2017

(54) WIRELESS SENSOR AND SYSTEM FOR MANAGING FETAL AND MATERNAL DATA OF SUBJECT

(71) Applicant: General Electric Company, Schenectady, NY (US)

(72) Inventor: Vijith Venugopalan, Bangalore (IN)

(21) Appl. No.: 15/306,276

(22) PCT Filed: May 22, 2015
(86) PCT No.: PCT/US2015/032233

§ 371 Date: Oct. 24, 2016

(30) Foreign Application Priority Data
May 30, 2014 (IN) 2671/CHE/2014

(51) Int. Cl. A61B 8/08; A61B 5/00; A61B 5/0402; A61B 8/00; A61B 8/02

(52) U.S. Cl. CPC A61B 8/0866; A61B 8/4254; A61B 8/02; A61B 8/4472; A61B 8/488; A61B 8/565; A61B 5/0402; A61B 5/4356; A61B 5/0022; A61B 2503/02

(57) ABSTRACT

A sensor of a maternal and fetal monitoring device is disclosed. The sensor includes a signal processor configured to transmit ultrasound beam on to a portion of a subject. Reflected ultrasound signals are received from the subject. These reflected ultrasound signals are processed to generate one or more of processed fetal information and processed maternal information. The sensor also includes a wireless module configured to communicate with a remote ultrasound processing system. The wireless module streams the processed fetal information and the processed maternal information to be stored in the remote processing subsystem.

### FIELD OF THE INVENTION

The subject matter disclosed herein relates to providing wireless sensor. More specifically the subject matter relates to a wireless sensor capable of communicating with a remote ultrasound processing system for managing fetal and maternal data.

### BACKGROUND OF THE INVENTION

Fetal monitoring involves monitoring of fetal condition during gestation and birth period. This process involves monitoring uterine activity and fetal heart rate. The fetal heart rate indicates whether the fetus is sufficiently supplied with oxygen. Most widely adopted technique used involves measuring a Doppler shift of an ultrasound signal reflected by a moving fetal heart. Another technique used may be an ultrasonic detection technique wherein an ultrasound transducer is placed externally on the pregnant women's abdomen and oriented such that the transmitted ultrasound waves impinge upon the fetal heart. The reflected ultrasound waves are received either by the same or by a different ultrasound transducer. The Doppler shift of the ultrasound wave is directly related to the speed of the moving parts of the heart, e.g. the heart valves and the heart walls.

The reflected ultrasound waves received at the ultrasound transducer are then sent to an ultrasound imaging subsystem for further processing to generate fetal data. The ultrasound transducer needs to be worn by the pregnant women and the ultrasound imaging subsystem needs to be within the vicinity of the ultrasound transducer. The ultrasound transducer may have a wired connection with the ultrasound imaging subsystem and thus makes movement of the pregnant women restrictive and process of monitoring the fetal data becomes a complex process. Further the medical practitioner or the doctor needs to visit the pregnant women's location to check the fetal data and so on which is time consuming and does not render the process efficient.

The doctor needs to have frequent visits to check the fetal data and the pregnant women needs to be in the hospital or the location where the ultrasound transducer and the ultrasound imaging subsystem are located. The pregnant women will find this highly inconvenient and risky as they may need to do frequent travels to location for fetal monitoring.

Accordingly, a need exists for an improved wireless sensor and method for managing fetal data and maternal data of a subject in a convenient manner.

### SUMMARY OF THE INVENTION

The object of the invention is to provide a wireless fetal sensor and system for managing fetal data and maternal data of a subject, which overcomes one or more drawbacks of the prior art. This is achieved by a wireless sensor having a wireless module for transmitting or streaming one or more of processed fetal information and processed maternal information as defined in the independent claim. The wireless sensor can be worn by a subject and obtain the processed fetal information from the fetus and the processed maternal information from the subject. The processed fetal information and/or the processed maternal information may be wirelessly communicated to a remote ultrasound processing system for further processing or storage. The processing involves converting the processed fetal information and the processed maternal information to fetal data and maternal data respectively. The fetal and maternal data can be examined by a medical practitioner to determine the health status of the fetus and the subject.

One advantage with the disclosed wireless sensor can be conveniently worn by the pregnant women in a convenient manner and its method of operation is also simple. The pregnant women can also move from one location to another without being near to a processing subsystem.

In an embodiment a sensor of a maternal and fetal monitoring device is disclosed. The sensor includes a signal processor configured to transmit ultrasound beam on to a portion of a subject. Reflected ultrasound signals are received from the subject. These reflected ultrasound signals are processed to generate one or more of processed fetal information and processed maternal information. The sensor also includes a wireless module configured to communicate with a remote ultrasound processing system. The wireless module streams the processed fetal information and the processed maternal information to be stored in the remote processing subsystem.

In another embodiment a system for managing at least one of fetal data and maternal data of a subject is disclosed. The system includes one or more sensors, wherein a sensor comprises a signal processor configured to transmit ultrasound beam on to a portion of a subject. Reflected ultrasound signals are received from the subject. These reflected ultrasound signals are processed to generate one or more of processed fetal information and processed maternal information. A wireless module in the fetal sensor is configured to stream the processed fetal information and the processed maternal information. A remote ultrasound processing system configured to wirelessly communicate with the one or more sensors. The remote ultrasound processing system is configured to receive the processed fetal information and the processed maternal information for storage.

In yet another embodiment a method of managing at least one of fetal data and maternal data of a subject is disclosed. The method includes transmitting ultrasound beam on to a portion of a subject using a sensor; receiving reflected ultrasound signals from the subject by the sensor; processing the reflected ultrasound signals to generate one or more of processed fetal information and processed maternal information; and streaming the processed fetal information and the processed maternal information to remote ultrasound processing system wirelessly communicable to the sensor, wherein the remote ultrasound processing system stores the processed fetal information and the processed maternal information.

A more complete understanding of the present invention, as well as further features and advantages thereof, will be obtained by reference to the following detailed description and drawings.

### BRIEF DESCRIPTION OF THE DRAWINGS

FIG. 1 is a schematic illustration of a pregnant patient utilizing fetal heart monitor;

FIG. 2 is a schematic illustration of fetal heart rate monitor in accordance with an embodiment;

FIG. 3 illustrates a sensor of a maternal and fetal monitoring device according to an embodiment;

FIG. 4 illustrates multiple sensors communicating with a remote ultrasound processing subsystem according to an embodiment;

FIG. 5 illustrates a system for managing fetal data and maternal data of a subject according to an embodiment;

FIG. 6 illustrates communication between a user device and another user device communicating with the remote ultrasound processing subsystem according to an embodiment; and

FIG. 7 illustrates a method of managing fetal data and maternal data of a subject according to an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical and other changes may be made without departing from the scope of the embodiments. The following detailed description is, therefore, not to be taken as limiting the scope of the invention.

As discussed in detail below, embodiments of the invention includes a sensor of a maternal and fetal monitoring device. The sensor includes a signal processor configured to transmit ultrasound beam on to a portion of a subject. Reflected ultrasound signals are received from the subject. These reflected ultrasound signals are processed to generate processed fetal information and processed maternal information. The sensor also includes a wireless module configured to communicate with a remote ultrasound processing system. The wireless module streams the processed fetal information and the processed maternal information to be stored in the remote processing subsystem.

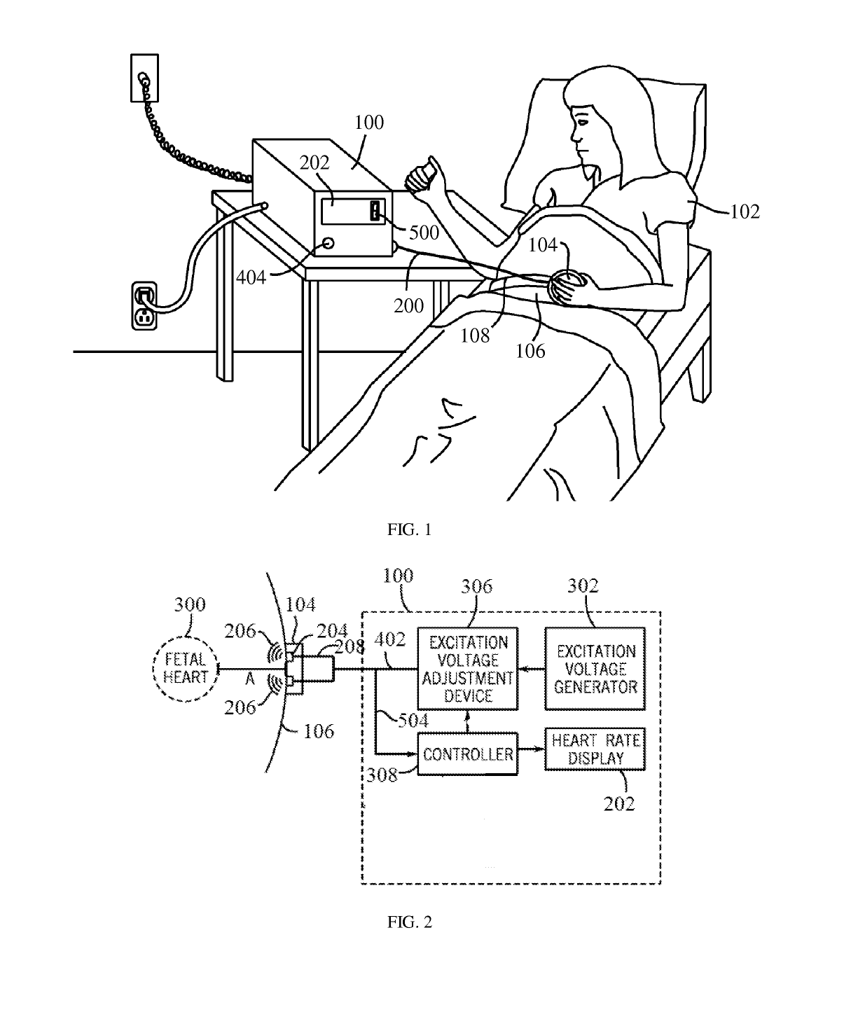

FIG. 1 illustrates a fetal heart rate monitor 100 that is commonly used to monitor the heart rate of the fetus of a pregnant patient 102. Although the fetal heart rate monitor 100 is shown in FIG. 1 in one exemplary form, it should be understood that the fetal heart rate monitor 100 could take many other forms while operating within the scope of the present disclosure.

In the embodiment of FIG. 1, the fetal heart rate monitor 100 includes a fetal sensor 104 i.e. an ultrasound probe that is secured to the patient's abdomen 106 by a strap 108. The fetal sensor 104 is shown in the embodiment of FIG. 1 as being coupled to the fetal heart rate monitor 100 by cable 200. As the fetal sensor 104 is connected using the cable 200 the patient 102 needs to be located close to the fetal heart rate monitor 100 to check the fetal heart rate and other health parameters. The health parameters include for example but not limited to SPO2, blood pressure, uterine activity, and pulse rate. However, it is contemplated that the fetal heart rate monitor 100 could communicate with the fetal sensor 104 using a wireless communication technique.

The fetal heart rate monitor 100 is shown in FIG. 1 as including a display screen 202 that typically displays the monitored heart rate of the fetus. The display screen 202 can be configured to display other monitored signals obtained from the patient 102.

During operation, when the fetal heart rate monitor 100 is powered on, one or more ultrasound transducers contained within the fetal sensor 104 each generate an ultrasound beam directed into the patient 102 through the skin of the abdomen. The fetal heart rate monitor 100 monitors the ultrasound signal returned to either the same or a different ultrasound transducer contained within the fetal sensor 104 to detect the beating of a fetal heart 300. Based upon data acquired from the fetal sensor 104, the fetal heart rate monitor 100 calculates the fetal heart rate and displays the calculated fetal heart rate on the display 202 in a known manner.

Referring now to FIG. 2, the detailed operation of the fetal heart rate monitor 100 will now be described. As illustrated in FIG. 2, the fetal sensor 104 is positioned on the exterior surface of the patient's abdomen 106. In the embodiment shown in FIG. 2, the fetal sensor 104 includes multiple ultrasound transducers 204. Each transducer 204 is operable to both generate an ultrasound beam 206 and receive reflected ultrasound energy from the fetal heart 300. In one embodiment of the disclosure, each of the ultrasound transducers 204 is a piezoelectric crystal that vibrates to create the ultrasound beam 206 emanating from the ultrasound transducer. The vibration of the piezoelectric crystal is created by an excitation voltage applied to the piezoelectric crystal through a voltage supply line 208.

Although in the embodiment shown in FIG. 2 each of the ultrasound transducers 204 is able to both transmit the ultrasound beam and receive the reflected ultrasound energy, the fetal sensor 104 could utilize separate transducers for transmitting and receiving the ultrasound energy.

During operation of the fetal heart rate monitor 100, the ultrasound transducers 204 generate the ultrasound beam 206 that penetrates the patient's abdomen 106 and travels into the pregnant patient until the ultrasound signal is reflected by beating fetal heart 300. As illustrated in FIG. 2, the distance A from the outer surface of the abdomen 106 to the fetal heart 300 must fall within the range of detection for the ultrasound transducers 204. The range of detection of the ultrasound transducers 204 is directly related to the signal strength of the ultrasound beam 206. In turn, the strength of the ultrasound beam 206 is directly related to the voltage level of the excitation voltage applied to the ultrasound transducers 204 along the voltage supply line 208. If the position of the fetal heart 300 is outside of the detection range of the ultrasound transducers 204, the fetal heart rate monitor 100 is unable to detect the heart rate of the fetus. In currently available fetal heart rate monitors, the value of the excitation voltage is selected such that the sensing distance of the ultrasound probe is sufficient to detect the fetal heart rate in a normal pregnant patient.

When the fetal heart rate monitor 100 is used with an obese patient, the distance A from the patient's abdomen 106 to the fetal heart 300 can be much greater than with a relatively thin or normal patient.

The fetal heart rate monitor 100 of the present disclosure includes circuitry that allows the power output, and thus the monitoring depth, of the sensor 104 to be selectively modified by a user. The selective modification of the power output of the sensor 104 allows the sensor 104 to detect the fetal heart rate at varying distances from the patient's abdomen 106. Further, the fetal heart rate monitor 100 may also allow an operator to control the amount of ultrasound power delivered to the pregnant patient.

The fetal heart rate monitor 100 includes an ultrasound excitation voltage generator 302. The excitation voltage generator 302 generates the typical excitation voltage that is used to drive the piezoelectric crystals that are incorporated into the ultrasound transducer 204. In prior fetal heart rate monitoring systems, the excitation voltage is applied directly to the ultrasound transducers 204. In such a prior art system, the excitation voltage level is fixed and cannot be modified by the user of the fetal heart rate monitor.

In the embodiment shown in FIG. 2, an excitation voltage adjustment device 306 is positioned between the excitation voltage generator 302 and the ultrasound transducers 204. The excitation voltage adjustment device 306 receives an excitation voltage along line and is operable to selectively amplify or reduce the excitation voltage as desired. The excitation voltage adjustment device 306 receives a voltage adjustment control signal from a controller 308 along a control line 400. In the embodiment illustrated, the controller 308 generates a control signal along line 400 that controls the voltage adjustment device 306 to selectively increase or decrease the excitation voltage from the excitation voltage generator 302. The modified excitation voltage from the voltage adjustment device 306 is provided to the ultrasound transducer 204 along the voltage supply line 402.

In the embodiment of the disclosure shown in FIG. 2, the controller 308 is a microprocessor that can generate digital signals along the control line 400 to the excitation voltage adjustment device 306. Although the controller 308 is shown as a microprocessor, the controller 308 could be a microcontroller, FPGA and CPLD while operating within the scope of the disclosure.

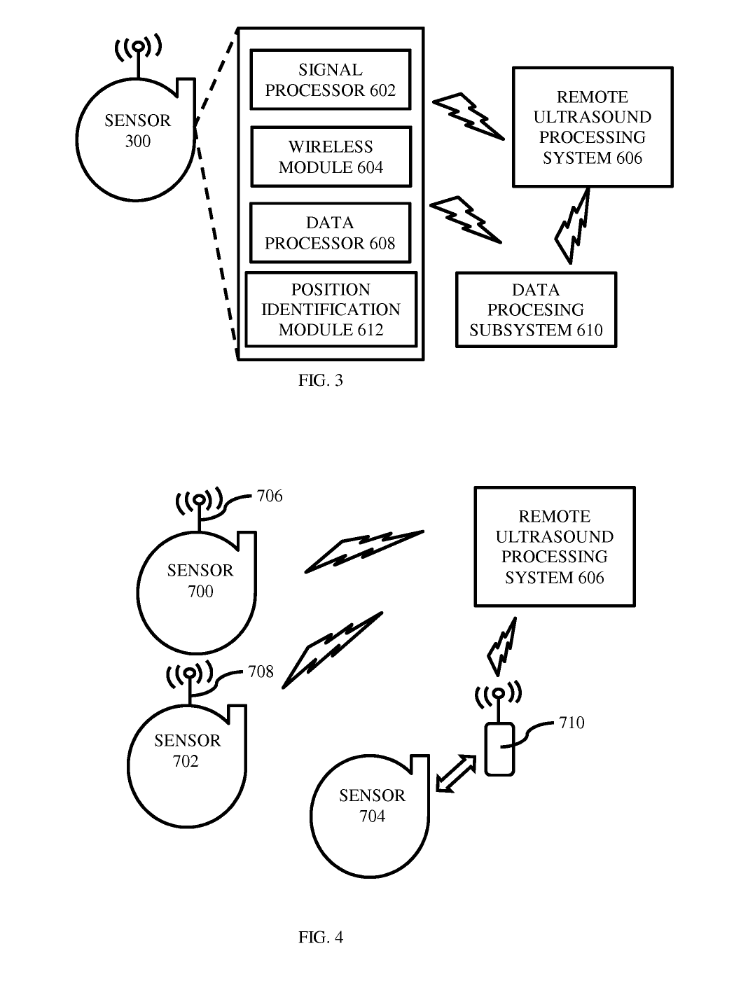

FIG. 3 illustrates a sensor 600 of a maternal and fetal monitoring device according to an embodiment. The sensor 600 is positioned on the patient's abdomen for monitoring health parameters of the fetus and the patient. The sensor 600 includes a signal processor 602 configured to transmit ultrasound beam on to a portion of the abdomen of the patient. Ultrasound signals are reflected from the subject. These reflected ultrasonic signals are received at the signal processor 602. The reflected ultrasonic signals are processed to generate one or more of processed fetal information and processed maternal information. In an embodiment the processed fetal information and processed maternal information may be raw data for example in a binary form, analog form or any other known forms. This is explained in detail in conjunction with FIGS. 1 and 2.

The sensor 600 includes a wireless module 604 configured to communicate with a remote ultrasound processing system 606. The remote ultrasound processing system 606 may be installed in a different location at a long distance away from the sensor 600. The remote ultrasound processing system 606 may be a combination of multiple servers that can be interconnected for storing the processed fetal data and maternal data received from different sources. The wireless module 604 is configured to transmit or stream the processed fetal information and the processed maternal information to the remote ultrasound processing system 606. In an embodiment the sensor 600 may have a wireless transmitter 608 for streaming the processed fetal information and the processed maternal information over a network (not shown in FIG. 3). The wireless transmitter 608 may be capable of operating through the network but are not limited to, a Local Area Network (LAN), a wireless LAN (WLAN), a Wireless Wide Area Network (Wireless WAN), a Wireless Personal Area Network (Wireless PAN), a Wireless Metropolitan Area Network (Wireless MAN), a Wireless Telecommunication Network, a 3^{rd} Generation communication (3G) network, a 4^{th} Generation communication (4G) network, public network (e.g. Internet), and a Long Term Evolution communication (4G LTE) network. As the sensor 600 has wireless capabilities the patient 102 (i.e. the subject) can move around while using the sensor 600 and the processed fetal information and/or the processed maternal information can be communicated to the remote ultrasound processing system 606 for further processing conveniently.

The remote ultrasound processing system 606 further processes the processed fetal information and the processed maternal information to generate fetal data and maternal data respectively. The fetal data may include fetal heart rate and values of other health parameters of the fetus. The maternal data may include maternal electrocardiography (ECG), maternal uterine activity, maternal pulse oximetry (SpO2), and maternal blood pressure. The fetal data and/or maternal data can be accessed from the remote ultrasound processing system 606 using any user device of a user. The user device may include but not limited to a laptop, a desktop computer, a computing device, a mobile device, a personal digital assistant (PDA). In another embodiment the sensor 600 includes a data processor 608 for processing the processed fetal information and the processed maternal information to generate the fetal data and the maternal data respectively. In this embodiment the fetal data may be transmitted to a data processing subsystem 610 having a display for presenting the fetal data and/or maternal data to the user.

In another embodiment the data processing subsystem 610 is configured to receive the processed fetal information and the processed maternal information from the sensor 600. Here the data processing subsystem 610 processes the processed fetal information and the processed maternal information to generate the fetal data and the maternal data. The fetal data and maternal data are then streamed to the remote ultrasound processing subsystem 606 for storage and retrieval. In yet another embodiment the fetal data and the maternal data are received from the remote ultrasound processing subsystem 606 by the data processing subsystem 610 and presented to the user through its display. The data processing subsystem 610 may be capable of wirelessly communicating with the sensor 600 and the remote ultrasound processing subsystem 606. Due to this wireless communication capability the patient 102 wearing the sensor 600 can be another location or at a distance from the data processing subsystem 610.

The sensor 600 may also include a position identification module 612 to identify position information associated with the sensor 600. The position information indicates the location of the patient 102. The position information may be global positioning system (GPS) coordinates.

Multiple sensors may be used for measuring or determining different health parameters associated with the fetus and the patient. These sensors may be ultrasound transducers, uterine contraction transducer, fetal ECG transducer and so on. FIG. 4 illustrates multiple sensors communicating with the remote ultrasound processing subsystem 606 according to an embodiment. As illustrated in FIG. 4, a sensor 700, a sensor 702 and a sensor 704 capable of communicating wirelessly with the remote ultrasound processing subsystem 606. The sensor 700 may be an ultrasonic transducer, the sensor 702 may be a uterine contraction transducer and the sensor 704 may be a fetal ECG transducer. The sensor 700 is used to obtain the position and other information of the fetus. The sensor 702 is used to determine the uterine activity of the fetus and the sensor 704 is used to measure maternal data of the patient.

The sensors 700-704 may be worn together by the patient 102 at a time. All the fetal information and maternal information from these sensors is wirelessly communicated to the remote ultrasound processing subsystem 606. In an embodiment the sensors 700-704 may communicate with a user device that can communicate with the remote ultrasound processing subsystem 606. The user device may be a laptop, a computing device, a mobile device, a personal digital assistant (PDA) and so on. The user device may have a user application that receives the processed fetal information and processed maternal information, and communicates to the remote ultrasound processing subsystem 606. The user application can be operated by the user for transferring the processed fetal information and the processed maternal information. The user application may be a light application that can connect the user device with the remote ultrasound processing subsystem 606. The sensor 700 and the sensor 702 may have wireless transmitter 706 and a wireless transmitter 708 respectively for transferring the processed fetal information to the user device. The user device and the sensors 700 and 702 may communicate over wireless communication techniques such as Bluetooth®, Zigbee®, wireless LAN and so on. In an alternate embodiment the user device and the sensors 700 and 702 are paired and thereafter communication is established. Further in another embodiment the processed fetal information and the processed maternal information transferred from the user device to the remote ultrasound processing subsystem 606 may be encrypted using any encryption techniques known in the art.

Further the sensor 704 may be connected to an external wireless transmitter 710 capable of communicating with the remote ultrasound processing subsystem 606 for streaming the processed maternal information. The external wireless transmitter 710 may operate based on various wireless technologies such as Bluetooth®, Zigbee®, wireless LAN and so on.

In an embodiment the sensors 700-704 may be also capable of wirelessly communicating with a data processing subsystem (such as the data processing subsystem 610) for transferring the processed fetal information and the processed maternal information to the data processing subsystem.

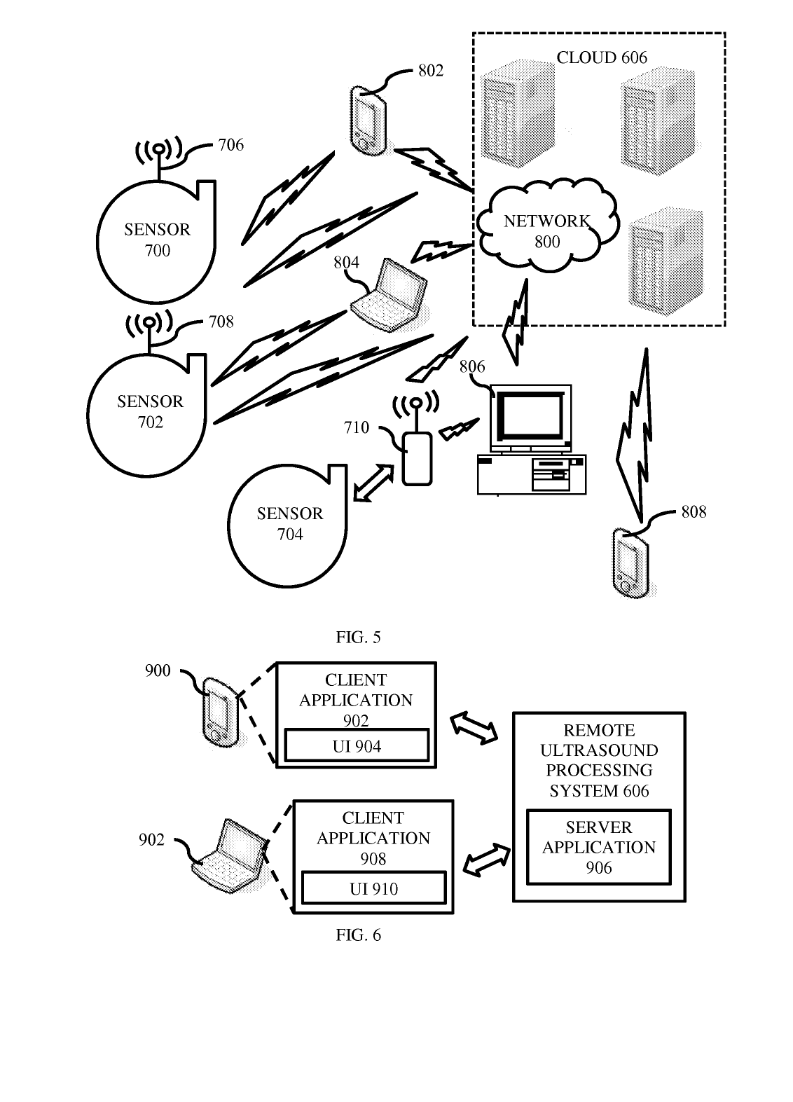

FIG. 5 illustrates a system 100 for managing fetal data and maternal data of a subject according to an embodiment. The system 100 includes multiple sensors such as the sensor 700, the sensor 702 and the sensor 704 capable of communicating the processed fetal data and the processed maternal data to the remote ultrasound processing subsystem 606. The remote ultrasound processing subsystem 606 may be a cloud based system. The cloud based system facilitates in providing services and comprises a network of interconnected nodes. An exemplary representation of the cloud based system as illustrated in FIG. 5 includes multiple servers connected to a network 800. Cloud based system include for example private clouds, public clouds, community clouds and hybrid clouds. In the case of a private cloud, infrastructure of this cloud is managed by an organization or a third party authorized by the organization. The private cloud may be step up in the office premises of the organization. Whereas in a public cloud, the infrastructure is managed by a company that provides cloud facility as a service to general public. Now in the case of community cloud, infrastructure of this cloud is supported by a particular group for example a group of companies needing a facility for sharing information. A hybrid cloud is a combination of multiple clouds such as a private cloud, a community cloud and a public cloud that work together based on standardized protocols that facilitate data flow between the different clouds and also in and out of each cloud. In a cloud based system, services may be offered to customers using multiple functional models for example, infrastructure as a service (IaaS), platform as a service (PaaS), software as a service (SaaS), network as a service (NaaS) and communication as a service (CaaS).

The sensor 700 wirelessly communicates with a mobile device 802. The sensor 700 transmits position and other information of the fetus to the mobile device 802. The mobile device 802 transfers the position and other information of the fetus to the remote ultrasound processing subsystem 606. Further the sensor 702 captures and transfers uterine activity data of the fetus through a laptop 804 to the remote ultrasound processing subsystem 606. The sensor 704 captures the maternal information of the patient and transfers to the remote ultrasound processing subsystem 606 through a computing device 806. The computing device 806 may be a desktop computer. The mobile device 802, the laptop 804 and the computing device 806 may be of the patient or a medical practitioner or a doctor and so on. The processed fetal information includes the position and other information of fetus, the uterine activity data and the fetal heart rate of the fetus. The processed maternal information includes maternal electrocardiography (ECG), maternal uterine activity, maternal pulse oximetry (SpO2), and maternal blood pressure.

In another embodiment the sensors 700-704 directly wirelessly communicate with the remote ultrasound processing subsystem 606 to stream the processed fetal information and the processed maternal information.

The processed fetal information and the processed maternal information are processed in the remote ultrasound processing subsystem 606 to generate the fetal data and the maternal data. The fetal data and maternal data can be accessed by a medical practitioner or the doctor through a user device 808. The user device 808 wirelessly communicates with the remote ultrasound processing subsystem 606 to retrieve the fetal data. The user device 808 may include a client application that enables the medical practitioner or the doctor to access the fetal data and the maternal data from the remote ultrasound processing subsystem 606.

FIG. 6 illustrates communication between a user device 900 and a user device 902 communicating with the remote ultrasound processing subsystem 606 according to an embodiment. In this figure only communication and processing of fetal information associated with the fetus is described for sake of convenience. Therefore it may be envisioned that another user device may be capable of streaming the processed maternal information to the remote ultrasound processing subsystem 606. The user device 900 may be used to transfer the processed fetal information obtained using a sensor to the remote ultrasound processing subsystem 606. The processed fetal information is obtained from fetus in the patient. The user device 900 has a client application 902 that provides a user interface (UI) 904 that enables the patient or a patient's attender to transfer the processed fetal data to the remote ultrasound processing subsystem 606. The UI 904 can be used to send request for connecting the user device 900 with the remote ultrasound processing subsystem 606. Thereafter a request may be send to the remote ultrasound processing subsystem 606 for transferring the processed fetal data. The remote ultrasound processing subsystem 606 also provides a server application 906 that can present the fetal data obtained from the processed fetal information. The server application 906 may be used by an administrator to access the fetal data in the remote ultrasound processing subsystem 606. The server application 906 is configured to process the requests for establishing connection and receiving the processed fetal data.

The fetal data can be obtained from the remote ultrasound processing subsystem 606, wherein a client application 908 in the user device 902 can be used. The client application 908 sends a request to obtain the fetal data which will be processed by the server application 906. The server application 906 processes the request and sends the fetal data. The client application 908 provides a UI 910 that presents the fetal data to the user of the user device 902. The user may be a medical practitioner or a doctor. The user device 902 may be in a different location. As a result the patient can use the sensor at home and send the processed fetal data to a remote ultrasound processing subsystem. A doctor can access the fetal data associated with the patient using a user device communicating with the remote ultrasound processing subsystem.

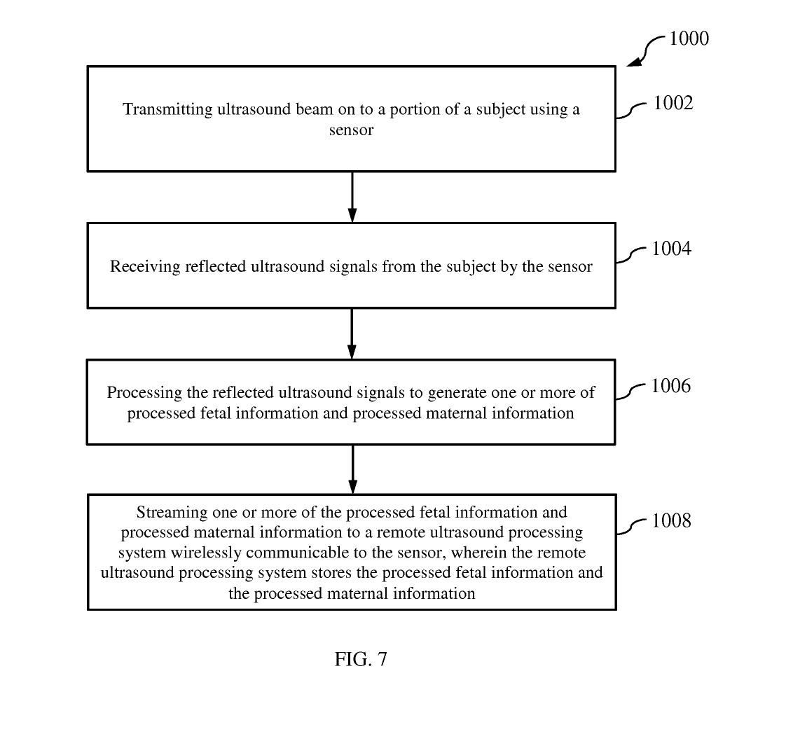

FIG. 7 illustrates a method 1000 of managing fetal data and maternal data of a subject according to an embodiment. The method 1000 includes transmitting an ultrasound beam on to a portion of the subject using a sensor at step 1002. Then at step 1004 ultrasonic signals reflected from the subject are received at the sensor. The reflected ultrasound signals are then processed to generate one or more processed fetal information and processed maternal information at step 1006. The processed fetal information and the processed maternal information are then streamed to a remote ultrasound processing system wirelessly communicable with the sensor at step 1008. The remote ultrasound processing system stores the processed fetal information and the processed maternal information and processes it to generate fetal data and maternal data. The remote ultrasound processing system can communicate with multiple user devices. The user devices may be used by the patient or patient's attender or a doctor or any medical practitioner. The user devices may be used to send the processed fetal information and the processed maternal information to the remote ultrasound processing system or to obtain the fetal data and the maternal data from the remote ultrasound processing system. The sensor may be worn by the patient and the patient moves around. Hence position information of the sensor may be monitored to identify the location of the patient.

From the foregoing, it will appreciate that the above disclosed a sensor capable of wirelessly transferring processed fetal information and processed maternal information to a remote ultrasound processing system. The remote ultrasound processing system then generates fetal data and maternal data from the processed fetal information and the processed maternal information. As the sensor has wireless capability the patient can wear the sensor and move around rather than being close to an data processing subsystem. Further the doctor can be at a remote location and can access the fetal data and maternal data which are retrieved from the data processing subsystem. Thus the doctor can give prescription or medical advises to the patient after analyzing the fetal data and the maternal data.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A sensor of a maternal and fetal monitoring device, the sensor comprising:
a signal processor configured to:
transmit ultrasound beam on to a portion of a subject;
receive reflected ultrasound signals from the subject; and
process the reflected ultrasound signals to generate at least one of processed fetal maternal information and processed maternal information; and
a wireless module configured to communicate with a remote ultrasound processing system, wherein the wireless module streams at least one of the processed fetal maternal information and the processed maternal information to be stored in the remote ultrasound processing system.

2. The sensor of claim 1, wherein the remote ultrasound processing system is configured to process at least one of the processed fetal information and the processed maternal information to generate at least one of fetal data and maternal data respectively of the subject.

3. The sensor of claim 1 further comprises a data processor configured to process at least one of the processed fetal information and the processed maternal information to generate at least one of fetal data and maternal data respectively of the subject.

4. The sensor of claim 3, wherein the wireless module is further configured to stream at least one of the processed fetal information and the processed maternal information to the remote ultrasound processing system for storage.

5. The sensor of claim 1, wherein the remote ultrasound processing system is a cloud based system.

6. The sensor of claim 1 further comprises a position identifying module configured to locate positional information associated with the sensor.

7. A system for managing at least one of fetal data and maternal data of a subject, wherein the system comprises:
at least one sensor, wherein a sensor of the at least one sensor comprises:
a signal processor configured to:
transmit ultrasound beam on to a portion of a subject;
receive reflected ultrasound signals from the subject; and
process the reflected ultrasound signals to generate at least one of processed fetal information and processed maternal information; and
a wireless module configured to stream at least one of the processed fetal information and processed maternal information; and
a remote ultrasound processing system configured to wirelessly communicate with the at least one sensor, wherein the remote ultrasound processing system is configured to receive at least one of the processed fetal information the processed maternal information for storage.

8. The system of claim 7, wherein the sensor further comprises a data processor configured to process at least one of the processed fetal information and the processed maternal information to generate at least one of fetal data and maternal data respectively of the subject.

9. The system of claim 8, wherein the wireless module is further configured to stream at least one of the fetal data and the maternal data to the remote ultrasound processing system.

10. The system of claim 8, wherein the wireless module is further configured to stream the processed fetal information and the processed maternal information to the remote ultrasound processing system.

11. The system of claim 7, wherein the remote ultrasound processing system is configured to process at least one of the processed fetal information and the processed maternal information to generate at least one of fetal data and maternal data respectively of the subject.

12. The system of claim 7, wherein the remote ultrasound processing system is configured to communicate with a plurality of user devices, wherein a user device of the plurality of user devices is capable of presenting at least one of the fetal data and the maternal data to a user.

13. The system of claim 7, wherein the remote ultrasound processing system is a cloud based system.

14. The system of claim 7 further comprises a data processing subsystem configured to:
receive at least one of the processed fetal information and processed maternal information from the signal processor; and
process the processed fetal information and processed maternal information to generate at least one of the fetal data and the maternal data respectively.

15. The system of claim 14, wherein the data processing subsystem is configured to transmit at least one of the processed fetal information and processed maternal information to the remote ultrasound processing system for storage.

16. A method of managing at least one of fetal data and maternal data of a subject, the method comprising:
transmitting ultrasound beam on to a portion of a subject using a sensor;
receiving reflected ultrasound signals from the subject by the sensor;
processing the reflected ultrasound signals to generate at least one of processed fetal information and processed maternal information; and
streaming at least one of the processed fetal information and the processed maternal information to a remote ultrasound processing system wirelessly communicable to the sensor, wherein the remote ultrasound processing system stores the processed fetal information.

17. The method of claim 16 further comprises processing at least one of the processed fetal information and the processed maternal information to generate at least one of the fetal data and the maternal data respectively of the subject by the remote ultrasound processing system.

18. The method of claim 16 further comprises streaming at least one of the fetal data and the maternal data to the remote ultrasound processing system for storage.

19. The method of claim 16 further comprises locating positional information associated with the sensor.

20. The method of claim 16 further comprises facilitating communication between the remote ultrasound processing system and a plurality of computing devices, wherein a computing device of the plurality of computing devices is capable of presenting the at least one of the fetal data and the maternal data to a user.
